(19) United States
(12) Patent Application Publication
(10) Pub. No.: US 2006/0127420 A1
(43) Pub. Date: Jun. 15, 2006

(54) Paclitaxel composition for the intravesical treatment of bladder tumor and preparation method thereof

(76) Inventors: Hesson Chung, Incheon (KR); Seo-Young Jeong, Gyeonggi-Do (KR); Ick-Chan Kwon, Seoul (KR); Yeong-Taek Park, Gyeonggi-Do (KR); In-Hyun Lee, Seoul (KR); Se-Wong Kim, Seoul (KR); Seung-Ju Lee, Seoul (KR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/521,695

(22) PCT Filed: Jul. 21, 2003
(86) PCT No.: PCT/KR03/01442

§ 371 Date: Nov. 3, 2005

(30) Foreign Application Priority Data
Jul. 20, 2002 (KR) 10-2002-0042792

(51) Int. Cl. A61K 31/337; A61K 9/00

(52) U.S. Cl. 424/400; 514/449

(57) ABSTRACT

The present invention relates to a paclitaxel composition and the preparation methods thereof for the treatment of bladder cancer wherein said paclitaxel composition comprises 4˜90% by weight of at least one selected from the monoglycerides, 0.01˜90% by weight of at least one oil, 0.01˜90% by weight of at least one emulsifier and 0.01˜20% by weight of paclitaxel. The composition of the present invention can treat bladder cancer effectively since the composition solubilizes paclitaxel, does not form aggregates, adsorbs well on the bladder wall and penetrates into the muscle layer of the bladder.

### TECHNICAL FIELD

The present invention relates to a paclitaxel composition for the treatment of bladder tumor and the preparation methods thereof.

### BACKGROUND ART

Bladder tumor is eleventh most frequently occurring cancer in the world and occupies 3˜4% of the all malignant tumor patients. Approximately there are more than 200,000 new patients each year, more than 150,000 patients among them are male. Bladder tumor can be divided into tree different categories; superficial, invasive and metastatic tumors.

Superficial bladder tumor is the tumor localized in the urothelium and the lamina propria, whereas invasive bladder tumor is the tumor that invaded into the muscle layer of the bladder but not metastasized to other parts of the body. Metastatic bladder tumor is the tumor that invaded to nearby organs, lymph nodes or other remote organs.

To treat superficial bladder tumor, transurethral resection (TUR) is performed in general. During TUR, a cystoscope is inserted into the bladder through the urethra. A tool with a small wire loop on the end of the cytoscope removes the cancer and to burn away any remaining cancer cells with an electric current. After TUR, patients may also have chemotherapy or biological therapy since the recurrence rate is 30˜85%. The most frequently used drug after TUR is Bacillus Calmette-Guerin (BCG) which is a tuberculosis vaccine. Also, anticancer drugs such as adriamycin or mitomycin C are administered once a week for 6˜8 weeks generally. If the preservation therapy fails or tumors are large and multiple, radical cystectomy can be performed.

For invasive bladder tumor, the most common type of surgery is radical cystectomy, which is performed when superficial tumor involves a large part of the bladder. Radical cystectomy is the removal of the entire bladder, the nearby lymph nodes, part of the urethra, and the nearby organs that may contain cancer cells. In men, the nearby organs that are removed are the prostate, seminal vesicles, and part of the vas deferens. In women, the uterus, ovaries, fallopian tubes, and part of the vagina are removed.

In some cases, the surgeon may remove only part of the bladder in a procedure called segmental cystectomy. This type of surgery is carried out when a patient has a low-grade cancer that has invaded the bladder wall in just one area, but this type for surgery is limited since there one has to make sure not to have cancer cells in other parts of urothelium. Also the recurrence rate is higher for segmental cystectomy than for radical cystectomy. For chosen patients, bladder preservation therapy along with transurethral resection, chemotherapy and radiation therapy are performed.

Radiation therapy or chemotherapy are the most frequently performed to treat metastatic bladder tumor. Cisplatin is known to be the most effective, and therefore combination therapy including cisplatin is generally chosen.

Since the patient loses the bladder after the radical cystectomy, studies are being carried out to preserve the bladder by chemotherapy and radiation therapy especially at the initial stage. To avoid the surgery, however, the size of tumor must be small without hydronephrosis. The most frequently used anticancer drugs are adriamycin, mitomycin C and cisplatin. In recent years, gemcitabine, paclitaxel or docetaxel is beginning to be used for the treatment of bladder tumor. Chemotherapy can be carried out alone or together with transurethral resection to treat superficial tumor. Intravesical chemotherapy is carried out once a week for several weeks by injecting the anticancer drug into the bladder through a urethral catheter. The injected anticancer drug is effective for several hours to affect the urothelium. After 30 year of clinical trials, as effective anticancer drugs for intravesical delivery, thiotepa and the related alkylating agent, ethoglucid, adriamycin and its derivative epirubicin and valrubicin, and mitomycin C have been selected. The recurrence rate reduces from 60% to 45% when intravesical chemotherapy is performed along with transurethral resection.

The anticancer drug, however, can cause side-effects. Myelosuppression due to systemic absorption of thiotepa, hypersensitivity by adriamycin and skin rash or genital rash by mitomycin C are the examples.

Recently paclitaxel and docetaxel are being used to treat baldder tumor in clinical trials. Paclitaxel shows excellent cytotoxicity to ovarian cancer, breast cancer, esophagus cancer, melanoma, leukemia, lung cancer, stomach cancer, prostate cancer, colon carcinoma, bladder cancer, lymphoidal tumor, hepatoma, tumor in central nervous system and brain tumor. Paclitaxel has been commercialized as intravenous injection Taxol® by Bristol-Myers Squibb Company. Paclitaxel is used in the form of emulsion preconcentrate (self-emulsifying system) due to its water-insolubility and therefore the solubilization technique has been developed along with the drug itself. One of the examples in the solubilization technique is the use of solubilizing agent for systemic administration such as intravenous injection. The above-mentioned Taxol® uses Cremophor EL (polyoxyethylene 35 castor oil), polyoxylethylated castor oil, polyoxyethoxylated castor oil and dehydrated alcohol, as solubilizing agents. Taxol® is a pre-concentrate type emulsion formulation that forms microemulsion spontaneously when dispersed in excess amount of water (U.S. Pat. No. 5,438,072). It is known, however, that solubilizing agent in Taxol® causes toxic side effects. Therefore, many studies have been performed to develop new paclitaxel formulations with high anticancer activity and low toxic effects. There are many patents describing, solid lipid nanoparticles, emulsion concentrate and emulsions prepared by using different oils and emulsifiers. Also other solubilization techniques by utilizing liposome, polymeric nanoparticles and polymeric micelles have been developed. These formulations solubilizing paclitaxel took advantage of the accumulated technological advancement already developed for other insoluble drugs. Also, even though paclitaxel is currently used to treat metastatic ovarian cancer and breast cancer, it is expected to be prescribed for various cancers, especially the metastatic solid tumors (lung cancer and hepatoma) in the near future. Therefore, market forecast is promising for paclitaxel.

From the pharmaceutical point of view, Taxol®, the most frequently prescribed paclitaxel formulation has a problem of forming precipitation when diluted inside the infusion bag due to the low solubility. In-line filter is used to prevent the precipitation from entering the blood stream of the patient. The exact dose of paclitaxel, therefore, is unknown and varies from time to time. Also, the plasticizer is known to leak out from the infusion bag made of PVC causing potential health problem. From the pharmacological point of view, Cremophor EL, the excipient can cause severe side-effects such as hypersensitivity, vasodilation, dyspnea, enervation and high blood pressure. From the pharmaceutical and pharmacological points of view, the stability and the safety of the drug must be improved by developing other administration routes and formulations.

Even though paclitaxel is cytotoxic against bladder tumor cells in vitro, Taxol® is ineffective in curing bladder tumor since the drug is hardly absorbed into the bladder cells in vivo. To treat bladder tumor by administering paclitaxel, therefore, it is imperative to develop new formulations that can solubilize and can help the absorption of paclitaxel into the bladder tissue.

In the mean time, monoolein also known as glyceryl monooleate is a monoglyceride that forms mucoadhesive liquid crystalline cubic phase in the presence of excess amount of body fluid. Even though it is possible to formulate bioadhesive paclitaxel formulations with monoolein, there is a potential danger to block the urinary track due to the high viscosity of the cubic phase. Therefore it is necessary to develop a new formulation that maintains the mucoadhesiveness but forms less viscous dispersion which can be discharged easily when mixed with urine. Also, bioavailability of the drug can only be achieved if paclitaxel does not precipitate out after the formulation forms dispersion in urine.

### SUMMARY OF THE INVENTION

Therefore, the object of the present invention is to provide a paclitaxel composition that does not form precipitations and can be absorbed effectively into the bladder tissue when administered intravesically and the preparation method thereof.

More particularly, the object of the present invention is to provide a paclitaxel composition that can be administered intravesically.

Also, another object of the present invention is to provide liquid formulation, semi-solid formulation that can be administered intravesically and the preparation method thereof.

### DETAILED DESCRIPTION OF THE INVENTION

The present invention provides oily paclitaxel composition for intravesical administration including at least one monoglyceride, at least one oil, at least one emulsifier and paclitaxel and the preparation method thereof.

Firstly, the present invention provides a paclitaxel composition for the treatment of bladder tumor.

More particularly, the above composition is composed of 4˜90% by weight of at least one monoglyceride, 0.01˜90% by weight of at least one oil, 0.01˜90% by weight of at least one emulsifier and 0.01˜20% by weight of paclitaxel (with respect to the total weight of the composition).

The above monoglycerides are selected from a group consisting of one or more saturated or an unsaturated monoglycerides having 10˜22 carbon atoms in the hydrocarbon chain. Monoglycerides is selected preferably from a group of consisting of monoolein, monopalmitolein, monomyristolein, monoelaidin and monoerucin, or semi-synthesized monoglycerides and their mixtures from triglycerides extracted from vegetable or animal oils, and more preferably monoolein.

The above oil is selected preferably from a group consisting of triglycerides, iodinated oil and vegetable or animal oil that can solubilize paclitaxel.

The above triglycerides are selected from a group consisting of one or more saturated or unsaturated triglycerides having 2˜20 carbon atoms in the hydrocarbon chain. For instance, triacetin, tributyrin, tricaproin, tricaprylin, tricaprin or triolein can be used.

The above iodized oils include iodized poppy seed oil such as Lipiodol, Ethiodol and iodized soybean oil.

The above vegetable oils include soybean oil, cottonseed oil, olive oil, poppyseed oil, linseed oil or sesame oil.

The above animal oils include squalane or squalene.

The above emulsifier is selected preferably from the group consisting of a phospholipid, a non-ionic surfactant, an anionic surfactant, a cationic surfactant, and bile acid.

The phospholipid is selected preferably from the group consisting of a phosphatidylcholine (PC) and its derivative, a phosphatidylethanolamine (PE) and its derivative, a phosphatidylserine (PS) and its derivative and a polymeric lipid wherein a hydrophilic polymer is conjugated to the lipid headgroup.

The non-ionic surfactant is selected from the group consisting of a poloxamer (also known as Pluronic: polyoxyethylene-polyoxypropylene copolymer), a sorbitan ester (Span), a polyoxyethylene sorbitan (Tween) and a polyoxyethylene ether (Brij).

The anionic surfactant is selected from the group consisting of a phosphatidylserine (PS) and its derivative, a phosphatidic acid (PA) and its derivative and sodium dodecyl sulfate (SDS).

The cationic surfactant is selected from the group consisting of 1,2-dioleyl-3-trimethylammonium propane (DOTAP), dimethyldioctadecylammonium bromide (DDAB), N-[1-(1,2-dioleyloxy)propyl]-N,N, N-trimethylammoniurn chloride (DOTMA), 1,2-dioleyl-3-ethylphosphocholine (DOEPC) and 3(-[N-[(N′,N′-dimethylamino)ethan]carbamoyl]cholesterol (DC-Chol).

The bile acid is selected from the group consisting of cholic acid, its salt and derivatives; deoxycholic acid, its salt and derivatives; chenocholic acid, its salt and derivatives; and lithocholic acid, its salt and derivatives.

Other additives can be added to the above composition including emulsifiers to be within 5% by weight. For instance, the composition can further comprise alcohol, polyol or Cremophor to improve the solubility of paclitaxel, tocopherol or tocopherol acetate to prevent oxidation, and fatty acid, fatty acid ester or fatty acid alcohol to increase drug absorption. Depending on the symptom, other insoluble drug can also be added in the composition including emulsifier according to the present invention.

The above insoluble drugs include other anticancer drugs and p-glycoprotein inhibitors.

The above other anticancer drugs include doxorubicin, cisplatin, carboplatin, carmustin (BCNU), dacarbazine, etoposide, 5-fluorouracil (5-FU), gemcitabine or paclitaxel derivatives. The above paclitaxel derivatives include docetaxel, bromotaxel and taxotere.

The above p-glycoprotein inhibitors include cinchonin, calcium channel blocker, calmodulin antagonist, Vinca alkaloid, antiarrhythmic, steroid, antihypertension drug, anthelmintic and immunosuppressant. The above calcium channel blockers include dihydropyridines such as verapamil, nifedipine, nicardipine and nitrendipine. The above calmodulin antagonists include trifluoroperazine. The above antihypertension drugs include reserpine. The above Vinca alkaloids include vincristine and vinblastine. The above steroids include progesterone. The above antiarrhythmics include amiodarone and quinidine. The above anthelmintics include quinacrine and quinine. The above immunosuppressants include cyclosporins, staurosporin and tacrolimus.

The above composition can be prepared by adding at least one monoglyceride, at least one oil, at least one emulsifier and paclitaxel at room or elevated temperature.

The method of preparing the above paclitaxel composition for the treatment of bladder tumor comprises the steps of;
1) preparing the viscous liquid by mixing 4˜90% by weight of at least one monoglyceride compound, 0.01˜90% by weight of at least one oil and, 0.01˜90% by weight of at least one emulsifier at temperature lower than 50° C. (step 1); and
2) preparing homogeneous mixture by solubilizing completely 0.01˜20% by weight of paclitaxel in said mixture in step (1) (step 2).

One of the examples in preparing the paclitaxel composition for the treatment of bladder tumor is as follows. In homogeneous viscous liquid obtained by mixing monoglyceride, oil and emulsifier at temperatures lower than 50° C., paclitaxel is added. The mixture was stirred or sonicated for 3˜5 minutes at temperatures lower than 50° C. to obtain homogeneous composition.

The method of preparing the above paclitaxel composition for the treatment of bladder tumor can also comprise the steps of;
1) preparing the paclitaxel solution by solubilizing 0.01˜20% by weight of paclitaxel in 0.01˜90% by weight of at least one oil by sonicating in a bath type sonicator (step 1); and
2) preparing homogeneous mixture by mixing the paclitaxel solution in step (1) and 0.01˜90% by weight of at least one emulsifier and 4˜90% by weight of monoglyceride (step 2).

The preparation methods described above are only two of many possible methods, and other preparation method can also be used to obtain the above paclitaxel composition for the treatment of bladder tumor.

The paclitaxel composition for the treatment of bladder tumor according to the present invention can be administered into the bladder to treat bladder tumor.

Particularly, it is preferable that the paclitaxel composition for the treatment of bladder tumor according to the present invention is delivered directly into the bladder via intravesical administration. It is more preferable that the paclitaxel composition for the treatment of bladder tumor according to the present invention is delivered directly into the bladder via intravesical administration after transurethral resection to treat superficial or invasive tumor.

The method of administering the above paclitaxel composition for the treatment of bladder tumor according to the present invention can comprise the steps of;

1) reducing the amount of remaining urine in the bladder to less than 10 ml (step 1); and

2) injecting 10˜100 ml of the paclitaxel composition for the treatment of bladder tumor according to the present invention into the bladder through urethral catheter and allowing the composition to stay inside the bladder for at least 2 hours (step 2).

The method of administering paclitaxel composition intravesically can also include a method of controlling the production rate of urine to 1 ml/min or less.

The above paclitaxel composition for the treatment of bladder tumor can be administered intravesically more than one time. Also, the above paclitaxel composition for the treatment of bladder tumor can be repeatedly administered intravesically for more than 6 weeks.

The above paclitaxel composition for the treatment of bladder tumor according to the present invention can be administered intravesically after transurethral resection to treat Ta, T1 or Tis tumor.

The paclitaxel composition for the treatment of bladder tumor according to the present invention exists as liquid, gel or semi-solid form depending on the composition at room temperature. Also the compositions of the present invention including paclitaxel are stable for a long period since the physical property of the composition does not change and the components do not degrade with time. Also the compositions for solubilization of insoluble drug of the present invention can be easily dispersed in water or in aqueous solutions to produce dispersion with particles bigger than 400 nm in diameter. Since the above dispersion of the composition does not form aggregation upon a long-time storage and can be adsorbed onto the bladder wall, the compositions of the present invention are efficient in solubilizing paclitaxel.

### BRIEF DESCRIPTION OF THE DRAWINGS

FIG. 1 is a graph showing the concentrations of paclitaxel, analyzed by HPLC, in urine and bladder tissue after intravesical administration of the paclitaxel composition for the treatment of bladder tumor in Example 1. One milliliter of the paclitaxel composition for the treatment of bladder tumor in Example 1 (corresponding to 6 mg of paclitaxel) was administered intravesically as an experimental group. For comparison, dispersion obtained by mixing 1 ml of the paclitaxel composition for the treatment of bladder tumor in Example 1 and 19 ml of water (dispersion control group, corresponding to 6 mg of paclitaxel) and emulsion obtained by mixing 1 ml of Taxol® of Bristol-Myers Squibb company and 19 ml of water (Taxol® group, corresponding to 6 mg of paclitaxel) were also administered intravesically.

FIG. 2 is a graph showing the changes in the concentration of paclitaxel as a function of the depth of the bladder tissue from urothelium to serosa, analyzed by HPLC, after intravesical administration of the paclitaxel composition for the treatment of bladder tumor in Example 1.
-●-; a group intravesically administered with 1 ml of the paclitaxel composition for the treatment of bladder tumor in Example 1 according to the present invention (Experimental group, 6 mg paclitaxel),
-◯-; a group intravesically administered with the dispersion obtained by mixing 1 ml of the paclitaxel composition for the treatment of bladder tumor in Example 1 and 19 ml of water (Dispersion control group, 6 mg paclitaxel), and
-▴- a group intravesically administered with the emulsion obtained by mixing 1 ml of Taxol® of Bristol-Myers Squibb company and 19 ml of water (Taxol® group, 6 mg paclitaxel).

FIG. 3 is a pathological photograph of bladder tissue 2 weeks after intravesical administration of the paclitaxel composition for the treatment of bladder tumor in Example 1 into the mice inoculated with bladder tumor cells.

a: a group intravesically administered with phosphate buffer solution, and

b: a group intravesically administered with 0.2 ml of the paclitaxel composition in Example 1 according to the present invention (1.2 mg paclitaxel).

FIG. 4 is a graph showing the weight of the bladder tissue 2 weeks after intravesical administration of the paclitaxel composition for the treatment of bladder tumor in Example 1 into the mice inoculated with bladder tumor cells.

Control group: a group intravesically administered with phosphate buffer solution and

Experimental group: a group intravesically administered with 0.2 ml of the paclitaxel composition in Example 1 according to the present invention (1.2 mg paclitaxel).

FIG. 5 is a graph showing the viability of bladder tumor cells after adding the dispersions of the paclitaxel composition in Example 1 according to the present invention diluted to paclitaxel concentrations of 0.1, 1, 10 μg/ml in phosphate buffer solution.

▪: a group treated with the paclitaxel composition in Example 1 and

□: a group treated with the control composition identical to the paclitaxel composition in Example 1 with the exception that the control composition does not contain paclitaxel.

### BEST MODE FOR CARRYING OUT THE INVENTION

This invention is explained in more detail based on the following Examples but they should not be construed as limiting the scope of this invention.

### EXAMPLE 1

### Paclitaxel Composition for the Treatment of Bladder Tumor According to the Change in the Composition (1)

(1) Manufacturing the Paclitaxel Composition for the Treatment of Bladder Tumor

Viscous oily solution was prepared by mixing completely 1 g monoolein, 0.5 g tricaprylin and 0.3 g of Tween 80 and warmed at 40° C. Paclitaxel (10.8 mg) was added into the oily solution and sonicated in a bath type sonicator for complete solubilization.

(2) Property Analysis of thus Prepared Composition for Solubilization of Paclitaxel

The size of the emulsion particles were measured by Photon Correlation Spectroscopy; QELS method) using Malvern Zetasizer (Malvern Instruments Limited, England) after diluting the emulsion by adding 3 mL of distilled water with 2 μL of thus obtained liquid formulation. An average particle size and polydispersity was obtained by measuring a given formulation three times (Orr, Encyclopedia of emulsion technology, vol. 1, 369-404, 1985). The polydispersity was obtained as the variance indicated by the logarithmic scale in the logarithmic normal distribution function. This method was used in measuring the particle size and the polydispersity throughout the following examples.

The composition was well dispersed in water with the average particle size of 600 nm. Paclitaxel precipitation was not observed under polarized light microscope 24 hours after preparing the dispersion, and phase separation was not observed either. The above composition exists as semi-solid or solid at room temperature and in the refrigerator, respectively, but as liquid at or above 40° C.

### EXAMPLE 2

### Paclitaxel Composition for the Treatment of Bladder Tumor According to the Change in the Composition (2)

The composition and dispersed liquid were prepared the same as those of the Example 1 with the exception that 1 g monoolein, 1 g tricaprylin, 0.4 g of Tween 80 and 14.4 mg of paclitaxel were used, and their particle size and polydispersity were measured by the same methods in the Example 1. Dispersion with the average particle size of 560 nm was obtained. Paclitaxel precipitation was not observed under polarized light microscope, and phase separation was not observed either. The above composition exists as semi-solid or solid at room temperature and in the refrigerator, respectively, but as liquid at or above 40° C.

The results of the Examples 1 and 2 are summarized in the following Table 1.

TABLE 1

| Content (weight %) | | | | Particle | |
|---|---|---|---|---|---|
| | | Tween | | size (nm) | Ex- |
| Monoolein | Tricaprylin | 80 | Paclitaxel | (polydispersity) | ample |
| 55.2 | 27.6 | 16.6 | 0.6 | 600 (0.200) | 1 |
| 41.4 | 41.4 | 16.6 | 0.6 | 560 (1.000) | 2 |

### COMPARATIVE EXAMPLE 1

### Paclitaxel Composition for the Treatment of Bladder Tumor without Oil (1)

The composition and dispersed liquid were prepared the same as those of the Example 1 with the exception that 1 g monoolein, 0.2 g of Tween 80 and 7.2 mg of paclitaxel were used, and their particle size and polydispersity were measured by the same methods in the Example 1. Dispersion with the average particle size of 670 nm was obtained. Paclitaxel precipitation was observed under polarized light microscope, and the dispersion became unstable 1 hour after preparation.

### COMPARATIVE EXAMPLE 2

### Paclitaxel Composition for the Treatment of Bladder Tumor without Oil (2)

The composition and dispersed liquid were prepared the same as those of the Example 1 with the exception that 1 g monoolein, 0.24 g of pluronic F 68 and 7.4 mg of paclitaxel were used, and their particle size and polydispersity were measured by the same methods in the Example 1. Dispersion with the average particle size of 630 nm was obtained. Paclitaxel precipitation was observed under polarized light microscope, and the dispersion became unstable 1 hour after preparation.

### COMPARATIVE EXAMPLE 3

### Paclitaxel Composition for the Treatment of Bladder Tumor without Monoolein (1)

The composition and dispersed liquid were prepared the same as those of the Example 1 with the exception that 1 g tricaprylin, 0.2 g of tween 80 and 7.2 mg of paclitaxel were used, and their particle size and polydispersity were measured by the same methods in the Example 1. Dispersion with the average particle size of 560 nm was obtained. Paclitaxel precipitation was not observed under polarized light microscope, and the dispersion was also stable without being phase-separated.

### EXAMPLE 3

### Paclitaxel Composition for the Treatment of Bladder Tumor According to the Change in the Oil (1)

The composition and dispersed liquid were prepared the same as those of the Example 1 with the exception that 1 g monoolein, 0.5 g tributyrin, 0.3 g of Tween 80 and 18 mg of paclitaxel were used, and their particle size and polydispersity were measured by the same methods in the Example 1. Dispersion with the average particle size of 950 nm was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

### EXAMPLE 4

### Paclitaxel Composition for the Treatment of Bladder Tumor According to the Change in the Oil (2)

The composition and dispersed liquid were prepared the same as those of the Example 1 with the exception that 1 g monoolein, 0.5 g lipiodol (Lipiodol Ultra-fluid, Laboratoire Guerbet, France, Iodine content: 38% by weight), 0.3 g of Tween 80 and 18 mg of paclitaxel were used, and their particle size and polydispersity were measured by the same methods in the Example 1. Dispersion with the average particle size of 680 nm was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

### EXAMPLE 5

### Paclitaxel Composition for the Treatment of Bladder Tumor According to the Change in the Oil (3)

The composition and dispersed liquid were prepared the same as those of the Example 1 with the exception that 1 g monoolein, 0.5 g squalane Sigma Chemical Company), 0.3 g of Tween 80 and 18 mg of paclitaxel were used, and their particle size and polydispersity were measured by the same methods in the Example 1. Dispersion with the average particle size of 598 nm was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

### EXAMPLE 6

### Paclitaxel Composition for the Treatment of Bladder Tumor According to the Change in the Oil (4)

The composition and dispersed liquid were prepared the same as those of the Example 1 with the exception that 1 g monoolein, 0.5 g safflower seed oil (Sigma Chemical Company), 0.3 g of Tween 80 and 18 mg of paclitaxel were used, and their particle size and polydispersity were measured by the same methods in the Example 1. Dispersion with the average particle size of 1040 nm was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

The results of the Examples 3˜6 are summarized in the following Table 2.

TABLE 2

| Oil * | Particle size (nm) (polydispersity) | Example |
|---|---|---|
| Tributyrin | 950 (0.661) | 3 |
| Lipiodol | 680 (1.000) | 4 |
| Squalane | 597 (0.550) | 5 |
| Safflower seed oil | 1040 (0.497) | 6 |

* Monoolein: Oil : Tween 80: Paclitaxel 55:28:16:1 (Weight ratio)

### EXAMPLE 7

### Paclitaxel Composition for the Treatment of Bladder Tumor According to the Change in the Paclitaxel Content (1)

The composition and dispersed liquid were prepared the same as those of the Example 1 with the exception that 1 g monoolein, 0.5 g tricaprylin, 0.3 g of Tween 80 and 36 mg of paclitaxel were used, and their particle size and polydispersity were measured by the same methods in the Example 1. Dispersion with the average particle size of 1450 nm was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

### EXAMPLE 8

### Paclitaxel Composition for the Treatment of Bladder Tumor According to the Change in the Paclitaxel Content (2)

The composition and dispersed liquid were prepared the same as those of the Example 1 with the exception that 1 g monoolein, 0.5 g tricaprylin, 0.3 g of Tween 80 and 54 mg of paclitaxel were used, and their particle size and polydispersity were measured by the same methods in the Example 1. Dispersion with the average particle size of 1630 nm was obtained. Paclitaxel precipitation was not observed under polarized light microscope, and phase separation was not observed either, 24 hours after preparing the dispersion. Unlike other compositions in Examples 1˜7, the above composition exists as liquid or solid at room temperature and in the refrigerator, respectively.

The results of the Examples 7-8 are summarized in the following Table 3.

TABLE 3

| Content (weight %) | | | | Particle | |
|---|---|---|---|---|---|
| | | Tween | | size (nm) | Ex- |
| Monoolein | Tricaprylin | 80 | Paclitaxel | (polydispersity) | ample |
| 55 | 27 | 16 | 2 | 1450 (1.000) | 7 |
| 54 | 27 | 16 | 3 | 1630 (1.000) | 8 |

### EXAMPLE 9

### Paclitaxel Composition for the Treatment of Bladder Tumor According to the Change in the Emulsifier

The composition and dispersed liquid were prepared the same as those of the Example 1 with the exception that Pluronic F68 (BASF Company) was used instead of Tween 80 and 18 mg of paclitaxel was used, and their particle size and polydispersity were measured by the same methods in the Example 1. Dispersion with the average particle size of 420 nm (polydispersity 0.284) was obtained. Paclitaxel precipitation was not observed under polarized light microscope, and phase separation was not observed either, 24 hours after preparing the dispersion. The above composition exists as semi-solid or solid at room temperature and in the refrigerator, respectively, but as liquid at or above 40° C.

### EXAMPLE 10

### In Vivo Intravesical Administration of Paclitaxel Composition for the Treatment of Bladder Tumor (Normal Rabbit)

The paclitaxel composition for the treatment of bladder tumor prepared in Example 1 was used for animal experiments.

\textcircled{1} Animal Model

New Zealand White Rabbits (8 weeks old) weighing 2.5˜3 kg were used regardless of gender.

\textcircled{2} Intravesical Administration of Paclitaxel Formulation

Sodium phentobarbital diluted to a concentration of 6 mg/ml was injected via intraperitoneal administration as a systemic anesthetic. Through the urethra, 10 Fr urethral catheter was inserted into the bladder and fixed subsequently by ballooning. Urine was completely discharged. As a control group 20 ml of diluted Taxol I® corresponding to 6 mg paclitaxel was administered intravesically. As an experimental group, 1 ml of the paclitaxel composition in the above Example 1 was administered (corresponding to 6 mg paclitaxel). Also, a dispersion prepared by mixing 1 ml of the paclitaxel composition in Example 1 and 19 ml water was also administered intravesically as a dispersion control group (corresponding to 6 mg paclitaxel). After the intravesical administration, the outer ends of the catheters were clamped to prevent the disharge of the administered drugs. After 2 hours, animals were sacrificed to determine the concentration of paclitaxel in the bladder, urine and blood.

\textcircled{3} Determination of Paclitaxel Concentration (HPLC Method)

Collected blood (200 μl) was added into a conical tube containing 20 μl of butyl paraben (100 μg/ml). After adding 0.5 ml of 35 mM ammonium acetate, paclitaxel was extracted by adding 4 ml tert-butylmethl ether. After centrifucation, organic solvent layer was collected and dried under reduced pressure.

In the dried residue, 100 μl of 60% acetonitril was added to dissolve the residue. Twenty microliters of the above sample was injected into HPLC to analyze the concentration of paclitaxel. Identical procedure was used for the urine to prepare the above blood HPLC samples. Bladder tissue was homogenized by ultrasonication after adding 1˜2 ml of 35 mM ammonium acetate buffer. Identical procedure was used for the homogenized bladder tissue to prepare above blood HPLC samples.

HPLC system consists of Hitachi HPLC, Hitachi L-7100 pump, Hitachi L-4200H UV-VIS detector and D-2500 integrator. The mobile phase was prepared by mixing identical amounts of 0.1% phosphate buffer solution (pH=6.86) and acetonitril at a flow rate of 1 ml/min and detected at 227 nm.

\textcircled{4} Paclitaxel Concentration in Blood, Urine and Bladder Tissue

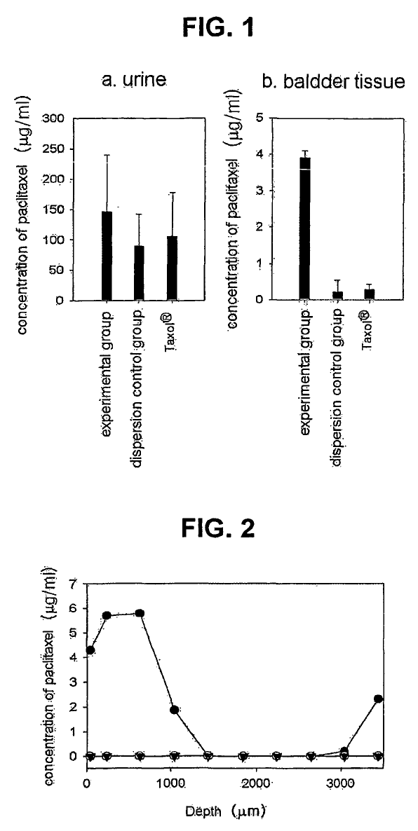

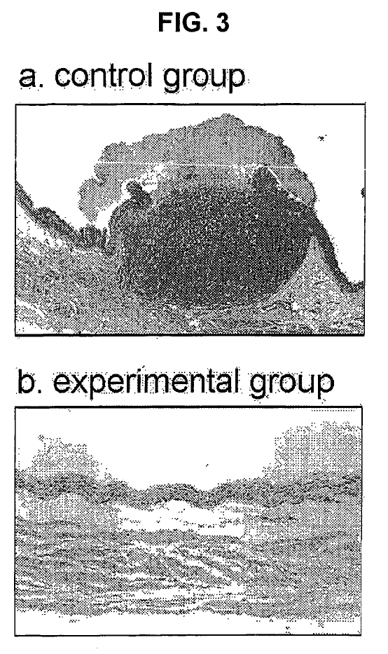

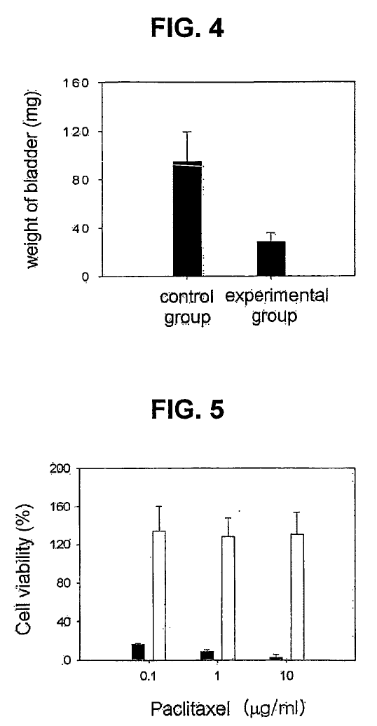

FIG. 1 is a graph showing the concentration of paclitaxel in urine and bladder tissue after intravesical administration of the paclitaxel composition for the treatment of bladder tumor in Example 1, dispersion control group and Taxol® group. Paclitaxel was not detected in the blood for the experimental and control groups. Paclitaxel concentration in urine for the experimental and control groups was 90˜150 μg/ml and did not show any statistical differences. Paclitaxel concentration in bladder tissue was 4 μg/mg for the experimental group, but those for the Taxol® and the dispersion control groups were less than 0.3 μg/mg.

\textcircled{5} Changes in the Concentration of Paclitaxel as a Function of the Depth of the Bladder Tissue

FIG. 2 shows the changes in the concentration of paclitaxel as a function of the depth of the bladder tissue from urothelium to serosa after intravesical administration of the paclitaxel composition for the treatment of bladder tumor in Example 1, dispersion control group and Taxol® group. The bladder tissue was sectioned to depths of 40 μm from the external transitional epithelium to internal smooth muscle layer. The outer most section of the transitional epithelium layer and the inner most section of the internal smooth muscle layer were discarded since they might be indirect contact with the paclitaxel formulation. Other sections in groups of 10 layers from out-to in sides were analyzed for the concentration of paclitaxel by HPLC as shown in FIG. 4.

Paclitaxel concentrations in the bladder tissue of the dispersion control group and Taxol® group were lower than the detection limit (0.5 μg/ml). On the other hand, paclitaxel penetrated into the parts of the smooth muscle cell in the bladder tissue when the paclitaxel composition for the treatment of bladder tumor of the present invention was administered.

### EXAMPLE 11

### In Vivo Intravesical Administration of Paclitaxel Composition for the Treatment of Bladder Tumor (Orthotopic Mouse Bladder Tumor Model)

The paclitaxel composition for the treatment of bladder tumor prepared in Example 2 was used for animal experiments.

\textcircled{1} Animal Model

C3H2 female mice (6˜8 weeks old) weighing 17˜20 mg were purchased from Korea Research Institute of Bioscience and Biotechnology and raised in groups of 5 animlas per chamber in a controlled environment of 20±1° C. in temperature and 50±10% in humidity fed by water and solid pressed meal.

\textcircled{2} Cultivation of Tumor Cells for Inoculation

MBT-2 (murine bladder tumor-2) cells, bladder cancer cell line, were cultivated in vitro in Dulbecco's modified Eagle's Medium (DMEM) supplemented with 10% fetal calf serum (FCS) and 1% penicillin/streptomycin at 37° C., 5% CO₂. After trypsinizing the tumor cells, they were mixed with DMEM which does not contain L-glutamin, FCS or antibiotics. The viability of the tumor cells was measured by trypan blue exclusion assay. The cells with higher than 90% viability were inoculated into the mice. The cells for the transplantation were diluted to 1×10⁶ per 1 ml.

\textcircled{3} Inoculation of Bladder Tumor Cells into the Mouse Bladder

Sodium phentobarbital diluted to a concentration of 6 mg/ml was injected via intraperitoneal administration as a systemic anesthetic. After shaving the back of the mouse, teflon intravenous catheter was placed inside the bladder through the urethra. ECG-electrode contact gel was applied on the back to place at the back-side down position, guide wire was inserted carefully, through the catheter until the end of the wire touches the bladder wall. After monopolar coagulation was performed for 5 s at the coagulation setting, the guide wire was eliminated. The tumor cell suspension (0.1 ml, 1×10⁶ cells/ml) was injected into the bladder. The outer end of the catheter was clamped so that the tumor cells can stay inside the bladder for at least an hour.

\textcircled{4} Intravesical Administration of Paclitaxel Formulation

Twenty four hours after injecting the tumor cells into the bladder, as a control group, 0.2 ml of phosphate buffered saline was administered intravesically (n=19) and, as an experimental group, 0.2 ml of the paclitaxel composition in the above Example 1 was administered (corresponding to 1.2 mg paclitaxel, n=3). After the intravesical administration, the outer ends of the catheters were clamped to prevent the discharge of the administered drugs. After 1 hour, animals were cultivated for 14 days under the conditions that they can freely eat, drink and urinate. The animals were sacrificed to observe the rate of tumor development, toxicity of the drug, and histopathological findings.

\textcircled{5} Statistical Analysis

Mann-Whitney-U-Test was carried out for statistical analysis and the data were considered significant when p-value was lower than 0.05.

\textcircled{6} Rate of Tumor Development

In the group of 19 control mice, 13 animals developed tumor (68.4%). On the other hand, in 3 mice of the experimental group, only one mouse developed a very small sized tumor (33.3%), which was significantly lower than the control group (p<0.05, Fisher's exact test). Bladder was fixed with formaldehyde and sectioned to prepare 6 μm thick samples. The obtained samples were stained with hematoxylin-eosin and observed with microscope. The results are shown in FIG. 3. In the control group, superficial bladder tumor cells were observed in the entire tissue, whereas normal tissue was observed for 2 mice and a small tumor was observed in one of the three mice.

\textcircled{7} Changes in the Weight of the Bladder

The average weights of the bladder in the control group and in experimental group were 94.8+24.5 mg and 28.8+6.7 mg, respectively (FIG. 4). Therefore the weight of the bladder in the group administered with the paclitaxel composition of the present invention was significantly lower than that in the control group (p<0.05, Mann-Whitney-U-Test),

\textcircled{8} Toxicity

The pathological findings in the kidney, liver, bone marrow and peripheral blood were identical for the group of mice administered with the paclitaxel composition of the present invention and the control group treated with PBS. Therefore, toxicity was not observed.

### EXAMPLE 12

### In Vitro Cytotoxicity of Paclitaxel Composition for the Treatment of Bladder Tumor

The paclitaxel composition for the treatment of bladder tumor prepared in Example 2 was used for animal experiments.

\textcircled{1} Cell Culture

MBT-2 (murine bladder tumor-2) cells, rat bladder cancer cell line, were cultivated in vitro in Dulbecco's modified Eagle's Medium (DMEM) supplemented with 10% fetal bovine serum (FBS) and 1% non-essential amino acid, 100 U/ml penicillin and 100 μg/ml streptomycin at 37° C.

\textcircled{2} Estimation of Cytotoxicity

After adding 100 μl of cell suspension at 5×10⁴ cell/ml into the wells of 96-well plate, the cells were cultured at 37° C. Twenty four hours after the incubation, the cell media were removed completely. The paclitaxel composition for the treatment of bladder tumor prepared in Example 2 was diluted and mixed with the media. The diluted compositions were applied to each well and cultivated for 24 hours. After removing the media containing the compositions, 100 μl of the fresh media was applied. Fifty microliters of MTT (3-[4,5-dimethylthiazol-2-yl]-2,5-diphenyltetrazolium bromide) solution prepared at 5 mg/ml in Hanks Balanced Salt Solution (HBSS) was applied to each well. After a 4-hour incubation in the dark at 37° C., the media were removed. The absorbance was determined at 570 nm after adding 200 μl of dimethylsulfoixde and 25 μl of glycine buffer solution at pH 10.5. The cell viability was determined by using the following formula.

$$Cell viability ( \% ) = \frac{Absorbance in the experimental group}{Absorbance in the control group} \times 100$$

The absorbance in the control group was obtained from the cells before applying diluted paclitaxel formulations. Cell viability at different paclitaxel concentrations are shown in FIG. 5. Cell viability was also determined by using the comparison group applied with the composition containing 1 g monoolein, 1 g tricaprylin and 0.4 g Tween 80. In case of the comparison group, the cell viability was 130% regardless of the concentrations indicating the composition is not toxic. On the other hand, the paclitaxel composition for the treatment of bladder tumor prepared in Example 2 shows higher toxicity proportional to the concentration of paclitaxel at the concentration range of 0.1˜10 μg/ml.

### INDUSTRIAL APPLICATION

The present invention provides a paclitaxel composition that can solubilize paclitaxel, does not from precipitation upon storage and is highly mucoadhesive. The pacltixel composition according to the present invention can kill tumor cells when administered via intravesical administration.

1. A paclitaxel composition for the treatment of bladder tumor via intravesical administration, comprising 4˜90% by weight of at least one monoglyceride compound, 0.01˜90% by weight of at least one oil, 0.01˜90% by weight of at least one emulsifier and 0.01˜20% by weight of paclitaxel.

2. The paclitaxel composition for the treatment of bladder tumor via intravesical administration according to claim 1, wherein said monoglyceride is chosen from saturated or unsaturated monoglyceride compounds having 10˜22 carbon atoms in the hydrocarbon chain.

3. The paclitaxel composition for the treatment of bladder tumor via intravesical administration according to claim 2, wherein said monoglyceride compound is chosen from monoolein, monopalmitolein, monomyristolein, monoelaidin, monoerucin, a mixture of monoglycerides semi-synthesized from triglycerides of vegetable and animal oil.

4. The paclitaxel composition for the treatment of bladder tumor via intravesical administration according to claim 1, wherein said oil is chosen from triglyceride, iodized oil, vegetable oil and animal oil.

5. The paclitaxel composition for the treatment of bladder tumor via intravesical administration according to claim 4, wherein said triglyceride is chosen from saturated and unsaturated triglycerides having 2˜20 carbon atoms in each hydrocarbon chain.

6. The paclitaxel composition for the treatment of bladder tumor via intravesical administration according to claim 4, wherein said triglyceride is chosen from triacetin, tributyrin, tricaproin, tricaprylin, tricaprin and triolein; wherein said iodized oil is chosen from Lipiodol, iodized poppy seed oil, Ethiodol and iodized soybean oil; wherein said vegetable oil is chosen from soybean oil, cottonseed oil, olive oil, poppyseed oil, linseed oil and sesame oil; and wherein said animal oil is chosen from squalane and squalene.

7.-9. (canceled)

10. The paclitaxel composition for the treatment of bladder tumor via intravesical administration according to claim 1, wherein said emulsifier is chosen from a phospholipid, a non-ionic surfactant, an anionic surfactant, a cationic surfactant and bile acid.

11. The paclitaxel composition for the treatment of bladder tumor via intravesical administration according to claim 10, wherein said phospholipid is selected from the group consisting of a phosphatidylcholine (PC) and its derivative, a phosphatidylethanolamine (PE) and its derivative, a phosphatidylserine (PS) and its derivative, and a polymeric lipid wherein a hydrophilic polymer is conjugated to the lipid headgroup; wherein said non-ionic surfactant is chosen from a poloxamer (Pluronic: polyoxyethylene-polyoxypropylene copolymer), a sorbitan ester (sorbitan esters; Span), a polyoxyethylene sorbitan (Tween) and a polyoxyethylene ether (Brij); wherein said anionic surfactant is chosen from a phosphatidylserine (PS) and its derivative, a phosphatidic acid (PA) and its derivative and sodium dodecyl sulfate (SDS); wherein said cationic surfactant is chosen from 1,2-dioleyl-3-trimethylammonium propane (DOTAP), dimethyldioctadecylammonium bromide (DDAB), N-[1-(1,2-dioleyloxy)propyl]-N,N,N-trimethylammonium chloride (DOTMA), 1,2-dioleyl-3-ethylphosphocholic acid (DOEPC) and 3β-[N-[(N′,N′-dimethylamino)ethan]carbamoyl]cholesterol (DC-Chol); and wherein said bile acid is chosen from cholic acid, its salt and derivatives; deoxycholic acid, its salt and derivatives; chenocholic acid, its salt and derivatives; and lithocholic acid, its salt and derivatives.

12.-15. (canceled)

16. The paclitaxel composition for the treatment of bladder tumor via intravesical administration according to claim 1, additionally comprising 0.01˜5% by weight of another additive.

17. The paclitaxel composition for the treatment of bladder tumor via intravesical administration according to claim 16, wherein the other additive chosen from Cremophor, tocopherol, tocopherol acetate, fatty acids, fatty acid esters, fatty acid alcohols, insoluble drugs, alcohols and polyols.

18. The paclitaxel composition for the treatment of bladder tumor via intravesical administration according to claim 17, wherein said insoluble drugs are chosen from anticancer drugs, p-glycoprotein inhibitors and hepatic metabolism blockers; wherein said alcohols are chosen from methanol, ethanol, propanol and isopropanol; and wherein said polyols are chosen from ethyleneglycol, propyleneglycol and polyethyleneglycol.

19. The paclitaxel composition for the treatment of bladder tumor via intravesical administration according to claim 18, wherein said anticancer drugs are chosen from doxorubicin, cisplatin, carboplatin, carmustin (BCNU), dacarbazine, etoposide, 5-fluorouracil and paclitaxel derivatives wherein said paclitaxel derivatives are chosen from docetaxel, bromotaxel and taxotere; wherein said p-glycoprotein inhibitors are chosen from cinchonins, calcium channel blockers, calmodulin antagonists, Vinca alkaloids, antiarrhythmics, steroids, antihypertension drugs, anthelmintics and immunosuppressants; and wherein said hepatic metabolism blockers are chosen from anticancer drugs including cyclosporin A, doxorubicin, etoposide (VP-16) and cisplatin; verapamil; and tamoxifen.

20.-21. (canceled)

22. The paclitaxel composition for the treatment of bladder tumor via intravesical administration according to claim 19, wherein said calcium channel blockers are dihydropyridines chosen from verapamil, nifedipine, nicardipine and nitrendipine; wherein calmodulin antagonist is trifluoroperazine: wherein antihypertension drug is reserpine: wherein Vinca alkaloids are chosen from vincristine and vinblastine: wherein steroid is progesterone: wherein said antiarrhythmics are chosen from amiodarone and quinidine: wherein said anthelmintics are chosen from quinacrine and quinine: and wherein said immunosuppressants are selected from the group consisting of cyclosporins, staurosporin and tacrolimus.

23.-32. (canceled)

33. A method of preparing the paclitaxel composition for the treatment of bladder tumor via intravesical administration according to claim 1, wherein said method comprises the steps of:
1) preparing the viscous liquid by mixing 4˜90% by weight of at least one monoglyceride compound, 0.01˜90% by weight of at least one oil and 0.01˜90% by weight of at least one emulsifier at temperatures lower than 50° C. (step 1); and
2) preparing homogeneous mixture by solubilizing completely 0.01˜20% by weight of paclitaxel in said mixture in step (1) (step 2).

34. The method of preparing the paclitaxel composition for the treatment of bladder tumor via intravesical administration according to claim 33, wherein the said mixture is heated to 50° C. in step (1) to speed up the solubilization process.

35. The method of preparing the paclitaxel composition for the treatment of bladder tumor via intravesical administration according to claim 33, wherein the said mixture is heated to 50° C. or sonicated in a bath type sonicator in step (2) to speed up the solubilization process.

36. A method of preparing the paclitaxel composition for the treatment of bladder tumor via intravesical administration according to claim 1, wherein said method comprises the steps of:
1) preparing the paclitaxel solution by solubilizing 0.01˜20% by weight of paclitaxel in 0.01˜90% by weight of at least one oil by sonicating in a bath type sonicator (step 1); and
2) preparing homogeneous mixture by mixing the paclitaxel solution in step (1) and 0.01˜90% by weight of at least one emulsifier and 4˜90% by weight of monoglyceride (step 2).

37. The method of preparing the paclitaxel composition for the treatment of bladder tumor via intravesical administration according to claim 36, wherein the said mixture is heated to 50° C. and sonicated in a bath type sonicator in step (2) to speed up the solubilization process.

38. The paclitaxel composition for the treatment of bladder tumor via intravesical administration according to claim 1, wherein said composition is administered intravesically after transurethral resection to treat superficial or invasive bladder tumor.

39. The paclitaxel composition for the treatment of bladder tumor via intravesical administration according to claim 1, wherein said composition is allowed to stay at least 2 hours after intravesical administration of 10˜100 ml through the urethral catheter after reducing the amount of urine to or less than 10 ml.

40. The paclitaxel composition for the treatment of bladder tumor via intravesical administration according to claim 39, wherein the method of controlling the production rate of urine to 1 ml/min or less additionally employed.

41. The paclitaxel composition for the treatment of bladder tumor via intravesical administration according to claim 39, wherein said composition is administered intravesically more than one time.

42. The paclitaxel composition for the treatment of bladder tumor via intravesical administration according to claim 39, wherein said composition is administered intravesically for more than 6 weeks.

43. The paclitaxel composition for the treatment of bladder tumor via intravesical administration according to claim 1, wherein said bladder tumor is Ta, T1 or Tis.
